(19) United States
(12) Patent Application Publication
(10) Pub. No.: US 2013/0190843 A1
(43) Pub. Date: Jul. 25, 2013

(54) PHOTO-STIMULATION METHOD AND DEVICE

(71) Applicants: Ming-Chieh TU, Keelung City (TW); Yi-Wei Hsiao, New Taipei City (TW); Chung-Pei Lee, Taipei City (TW); Jung-Chien Chang, New Taipei City (TW); Yu-Chia Tsao, Taipei City (TW)

(72) Inventors: Ming-Chieh TU, Keelung City (TW); Yi-Wei Hsiao, New Taipei City (TW); Chung-Pei Lee, Taipei City (TW); Jung-Chien Chang, New Taipei City (TW); Yu-Chia Tsao, Taipei City (TW)

(73) Assignee: Forward Electronics Co., Ltd., Taipei City (TW)

(21) Appl. No.: 13/470,559

(22) Filed: May 14, 2012

(30) Foreign Application Priority Data
Jan. 19, 2012 (TW) 101102136

(51) Int. Cl. A61N 5/06

(52) U.S. Cl. 607/90; 607/88

(57) ABSTRACT

Disclosed is a photo-stimulation method and device. The method includes the following steps: providing a light-emitting diode (LED) illuminant which is a yellow, red, or blue LED; and illuminating a subject by the LED illuminant to promote collagen synthesis, to suppress microbial growth, or to inhibit melanin synthesis, wherein the yellow LED is in an illuminance range from 1,000 to 3,500 lux, the red LED is in an illuminance range from 6,000 to 9,500 lux, and the blue LED is in an illuminance range from 3,000 to 7,000 lux.

### CROSS REFERENCE TO RELATED APPLICATION

This application claims the benefits of the Taiwan Patent Application Serial Number 101102136, filed on Jan. 19, 2012, the subject matter of which is incorporated herein by reference.

### BACKGROUND OF THE INVENTION

1. Field of the Invention

The present invention relates to a photo-stimulation method and a photo-stimulation device and, more particularly, to a photo-stimulation method and a photo-stimulation device which can promote collagen synthesis, enhance inhibition of bacterial growth, or inhibit melanin synthesis. 2. Description of Related Art

After dermatological diagnosis, drugs are generally used to treat patients' skin conditions, such as acne. However, drug therapy frequently incurs side effects and long-term drug administration also results in metabolic loads on patients. Notably, such therapy does not bring desirable efficacy of treatment and treated patients often have a relapse of skin conditions. Hence, patients' skin conditions can not be efficiently eradicated.

In recent years, medical cosmetology has been greatly developed. Some research reported that blue light with a wavelength of 400-475 nm could be applied to treat acne. Because photosensitive coproporphyrin of Propionibacterium acnes or tissue cells is reacted with blue light to form toxic singlet oxygen and free radicals which kill bacteria and partial sebaceous gland, inflammation and red and swollen conditions of tissues caused by acne can be alleviated. In addition, red light with a wavelength of 600-750 nm, yellow light with a wavelength of 550-600 nm, and green light with a wavelength of 500-570 nm can stimulate fibroblasts in the dermis to induce synthesis of collagen and to prevent skin aging.

At present, in order to achieve the above-mentioned effects, laser or intense pulsed light is often applied in the industry of medical cosmetology. However, owing to high energy and intensity of the light, it is easy for the aforesaid light to cause injury to cells. General light sources or light-emitting diodes (LEDs) have been recently developed to replace the high-intensity light above. Due to the relatively low energy of light emitted from LEDs, appropriate illuminance of the light needs to be found to achieve the aforesaid effects. Too low illuminance of light does not induce good treatment and, conversely, too high illuminance of light injures cells and has to be generated by large LED devices. Accordingly, it is difficult to create a compact and portable LED device for phototherapy.

Therefore, it is desirable to provide a photo-stimulation method and device in which LEDs are adjusted in a specific range of illuminance to promote collagen synthesis, to suppress microbial growth, or to inhibit melanin synthesis so that labor, power, and time costs can be economized and the treated patients' skin condition can be improved.

### SUMMARY OF THE INVENTION

The object of the present invention is to provide a photo-stimulation method in which LEDs are set to emit red or yellow light in a specific range of illuminance so as to stimulate collagen synthesis of fibroblasts and to promote blood circulation as well as speed up removal of dead cells. Additionally, in the photo-stimulation method, LEDs may be set to emit blue light in a specific range of illuminance to inhibit or kill P. acnes or to suppress or reduce melanin synthesis of melanocytes.

To achieve the object, one aspect of the present invention provides a photo-stimulation method including the following steps: providing a light-emitting diode (LED) illuminant which is selected from a group consisting of a yellow LED, a red LED, and a blue LED; and illuminating a subject by the LED illuminant to promote collagen synthesis, to suppress microbial growth, or to inhibit melanin synthesis, wherein the yellow LED is in an illuminance range from 1,000 to 3,500 lux, the red LED is in an illuminance range from 6,000 to 9,500 lux, and the blue LED is in an illuminance range from 3,000 to 7,000 lux.

In conventional methods, laser or intense pulsed light with different wavelengths are applied to treat acne and to stimulate fibroblasts of dermis for an increase of collagen synthesis. Because laser or intense pulsed light with high intensity has to be produced by large apparatuses, it is difficult for general consumers to have such large apparatus. Although there has been research for LEDs which are used as a light source for acne treatment and promotion of collagen synthesis, the influence of the illuminance of LEDs on cells or bacteria is not studied in conventional research. Therefore, it is not confirmed that the conventional method can achieve the aforesaid effects with unspecified illuminance of light. On the contrary, in the method of the present invention, blue, yellow or red light of LEDs used as illuminants is adjusted in corresponding ranges of illuminance to efficiently achieve stimulation of fibroblasts, promotion of collagen synthesis, inhibition or killing of P. acnes, and reduction and inhibition of melanin synthesis.

When sustained illumination is achieved with yellow or red LEDs at appropriate illuminance for a suitable period of time, macrophages are stimulated to secrete cytokines for stimulation of fibroblast division. Meanwhile, fibroblasts are stimulated to synthesize DNA and secrete fibroblast growth factors (FGFs) for collagen synthesis. If the subject is a cell in vivo, for example, a fibroblast in dermis or a macrophage, light illumination on the skin can directly induce wound healing and anti-aging effects. Alternatively, if the subject is a cell in vitro, the cell can be treated as mentioned above and then implanted into animals for the aforesaid benefits. Accordingly, the subject of the present invention refers to a photo-stimulated subject.

In the photo-stimulation method of the present invention, when the LED illuminant is a yellow or red LED, the subject is preferably a fibroblast, a macrophage, or a combination thereof In a preferred example of the present invention, the subject is a fibroblast. Furthermore, the wavelength of the yellow LED can range from 570 to 590 nm and the wavelength of the red LED can range from 620 to 750 nm. The illuminating time of the yellow or red LED is not limited as long as the aforesaid benefits occur in the subject and the light illumination is not harmful thereto. The illuminating time can be adjusted according to the predetermined illuminance of light emitted from the yellow and red LEDs. If the light illuminance is relatively high, the benefits can be achieved in a relatively short period of illuminating time. On the contrary, if the light illuminance is relatively low, the benefits can be carried out over a relatively long period of illuminating time.

For example, in an illuminance range of red LEDs light from 6,000 to 9,500 lux or in an illuminance range of yellow LEDs light from 1,000 to 3,500 lux, the illuminating time can range from 5 to 90 min. When light of a red LED at 9,890 lux, beyond the aforementioned range of illuminance, is used for illumination, the subject is not influenced by light illumination for a short period of time but is injured by light illumination for a long period of time due to an overdose of illuminance. On the contrary, when light of a red LED at 6,000 lux or less is used for illumination, the benefits are not achieved even under light illumination for a long period of time.

When sustained illumination is achieved with blue LEDs at appropriate illuminance for a suitable period of time, melanocytes are stimulated to directly or indirectly influence tyrosinases so as to reduce synthesis of melanin and thus avoid precipitation of melanin. Similarly, photosensitive coproporphyrin of P. acnes is reacted with blue light to form cytotoxic singlet oxygen which damages or further kills bacteria. In addition, partial sebaceous gland is also influenced to reduce sebum secretion. If the subject is skin, acne pocks or P. acnes on the skin surface can be killed under illumination of blue light. Alternatively, if the subject is a melanocyte on a skin surface, melanin synthesis can be inhibited to avoid an increase in skin chrominance and thus achieve skin whitening

In the photo-stimulation method of the present invention, when the LED illuminant is a blue LED, the subject is P. acnes, a melanocyte, or a combination thereof Furthermore, the wavelength of the blue LED can range from 450 to 475 nm. The illuminating time of the blue LED is not particularly limited as long as the aforesaid benefits occur in the subject and the light illumination is not harmful thereto. The illuminating time can be adjusted according to the predetermined illuminance of light emitted from the blue LEDs. If the light illuminance is relatively high, the benefits can be achieved in a relatively short period of illuminating time. Conversely, if the light illuminance is relatively low, the benefits can be carried out in a relatively long period of the illuminating time.

For example, in an illuminance range of blue LEDs light from 3,000 to 7,000 lux, the illuminating time can range from 5 to 90 min. When blue light beyond the aforementioned range of illuminance is used for illumination, the subject is not influenced by light illumination for a short period of time but is injured by light illumination for a long period of time due to an overdose of illuminance. When blue light lower than the illuminance range is used for illumination, the benefits are not achieved even under light illumination for a long period of time. In a preferred example of the present invention, at 5,330 lux of light from a blue LED, light illumination for 30 minutes or more can achieve induction of melanin synthesis. In another preferred example of the present invention, at 5,710 lux of light from a blue LED, light illumination for more than 10 minutes can achieve inhibition of P. acnes and reduction of melanin synthesis in melanocytes.

Another object of the present invention is to provide a photo-stimulation device. In the device, LEDs are set to emit red or yellow light in a specific range of illuminance so as to stimulate collagen synthesis of fibroblasts and to promote blood circulation as well as speed up removal of dead cells. Alternatively, in the photo-stimulation device, LEDs emit blue light in a specific range of illuminance to inhibit or kill P. acnes or to reduce or suppress melanin synthesis of melanocytes.

In order to achieve the object, another aspect of the present invention provides a photo-stimulation device which includes: a casing forming a deposition space and having a top surface and a lateral surface, wherein the top surface is provided with a light-output window; a diffuser plate covering the light-output window of the casing; a first illuminant module located in the deposition space of the casing and having a first light-emitting diode (LED) located under the diffuser plate, and the first light-emitting diode selected from a group consisting of a red LED, a yellow LED, and a blue LED, wherein the light emitted from the yellow LED and passing through the diffuser plate has an illuminance in a range of 1,000-3,500 lux, the light emitted from the red LED and passing through the diffuser plate has an illuminance in a range of 6,000-9,500 lux, and the light emitted from the blue LED and passing through the diffuser plate has an illuminance in a range of 3,000-7,000 lux; and a controller module electrically connected with the first illuminant module and a power module.

In the photo-stimulation device of the present invention, blue, yellow, or red LEDs are used as illuminants and light with different colors is set in corresponding ranges of illuminance. Once a subject is illuminated by the photo-stimulation device of the present invention, fibroblasts can be stimulated to synthesize collagen, P. acnes can be inhibited or killed, and melanin synthesis in melanocytes can be suppressed and reduced.

In the photo-stimulation device of the present invention, the power module can be an external power supply or be placed in the deposition space of the casing. The power module can contain rechargeable or dry batteries or microbatteries placed in the deposition space of the casing. Alternatively, if the power module is an external power supply or a rechargeable battery placed in the deposition space of the casing, the controller module can selectively further include a charge socket that provides an electrical connection between the power module and the controller module.

In the photo-stimulation device of the present invention, the controller module can selectively comprise a power switch mounted on the surface of the casing to control power output of the power module. Furthermore, the casing is preferably made of a material with low transmittance, for example, a material with high reflectivity or density such that light leakage of the photo-stimulation device can be prevented. Also, in order to prevent light leakage of the photo-stimulation device, one skilled in the art of the present invention can increase tightness of the whole device by various structural designs.

In the photo-stimulation device of the present invention, the lateral surface of the casing can be selectively provided with a light-output hole. In this case, the photo-stimulation device can further include a light-transmission plate covering the light-output hole, and a second illuminant module deposed corresponding to the light-transmission plate and emitting light which passes through the light-transmission plate. In this case, the controller module can further include a mode switch mounted on the surface of the casing to turn on the first illuminant module or the second illuminant module, i.e. to switch between the first illuminant module and the second illuminant module. In the first and second illuminant modules, assigned LEDs can be in the same color or different colors.

In the photo-stimulation device of the present invention, the diffuser plate placed on the light-output window is beneficial for uniform light emission and to avoid direct light illumination on users' eyes. Also, uniformity of photo-stimulation of the device can be increased. In other words, light of the LEDs is classified into a point source passing through the diffuser plate and then forming a surface light at the light-output window. The light-transmission plate placed on the light-output hole does not have to be a diffuser plate. If the light-transmission plate is a diffuser plate, the benefits described above can be achieved. If the light-transmission plate is not a diffuser plate, light supplied by a point light source can be directly transmitted.

In the photo-stimulation device of the present invention, the first and second illuminant modules can be designed as being electable. In other words, red, yellow and blue LEDs constitute the first and second illuminant modules. If there is a need of red light illumination, the illuminant module that is constituted by red LEDs is arranged in the device. If there is a need of blue light illumination, the illuminant module that is constituted by blue LEDs is arranged in the device. Furthermore, LEDs used in the first and second illuminant modules can be designed as being replaceable. In other words, if there is a need of yellow light illumination, LEDs used in the first or second illuminant module can be replaced with a yellow LED.

In conclusion, the photo-stimulation method and device can employ LEDs with different colors such as yellow, red, and blue for photo-stimulation. Therefore, inhibition or killing of P. acnes, reduction or suppression of melanin synthesis in melanocytes, and promotion of collagen synthesis can be achieved so as to treat acne pocks and carry out skin whitening or anti-aging benefits.

### BRIEF DESCRIPTION OF THE DRAWINGS

FIG. 1 is a perspective view of a photo-stimulation device in Example 1 of the present invention;

FIG. 2 is a side view of a photo-stimulation device in Example 1 of the present invention;

FIG. 3 is a system block diagram of a photo-stimulation device in Example 1 of the present invention;

FIG. 4 is a chart of human fibroblast viability in Example 2 of the present invention;

FIG. 5 is a chart of human fibroblast viability in Example 3 of the present invention;

FIG. 6 is a chart of collagen synthesis in Example 4 of the present invention;

FIG. 7 is a chart of human fibroblast viability in Example 5 of the present invention;

FIG. 8 is a chart of human fibroblast viability and collagen synthesis in Example 6 of the present invention;

FIG. 9 is a chart of human melanocyte viability in Example 7 of the present invention;

FIG. 10 is a chart of melanin synthesis in Example 8 of the present invention;

FIG. 11 is a chart of P. acnes viability in Example 9 of the present invention; and

FIG. 12 is a chart of human fibroblast viability in Comparative Example of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### EXAMPLE 1

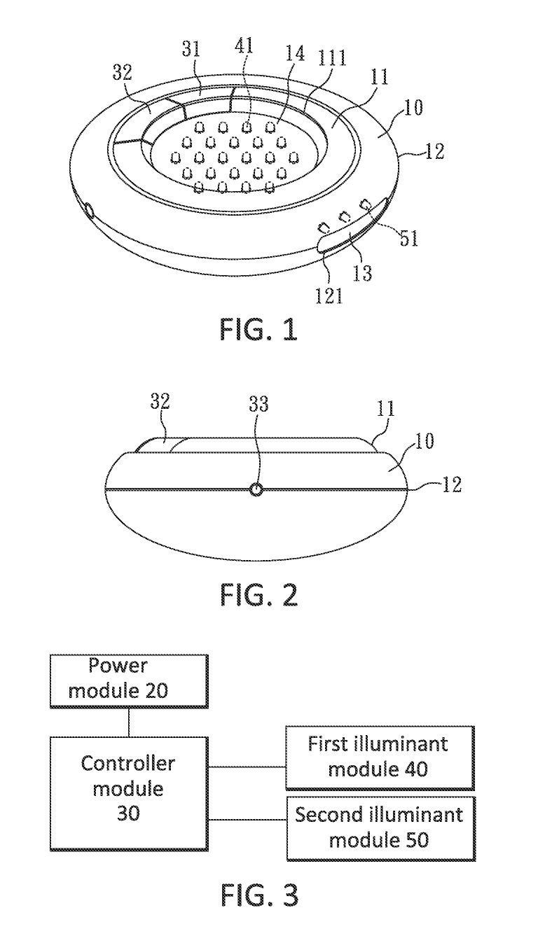

With reference to FIGS. 1 to 3, FIGS. 1 to 3 show a perspective view, a side view, and a system block diagram of a photo-stimulation device of the present invention, respectively.

As shown in FIGS. 1 to 3, the photo-stimulation device of the present invention includes: a casing 10, a diffuser plate 14, a light-transmission plate 13, a first illuminant module 40, a second illuminant module 50, and a controller module 30.

The casing 10 forms a deposition space for receiving different modules. In addition, the casing 10 has a top surface 11 and a lateral surface 12. The top surface 11 is provided with a light-output window 111. The lateral surface 12 is provided with a light-output hole 121.

The light-output window 111 of the top surface 11 is covered by the diffuser plate 14, and the light-output hole 121 of the lateral surface 12 is covered by the light-transmission plate 13. The second illuminant module 50 corresponds to the light-transmission plate 13 and is placed in the deposition space of the casing 10. The second illuminant module 50 emits light passing through the light-transmission plate 13 and has one or more second LEDs 51. Herein, if the light-transmission plate 13 is used for light transmittance but not for light diffusion, the second illuminant module 50 serves as a point source of light.

The first illuminant module 40 is located in the deposition space of the casing 10 and a plurality of first LEDs 41 are arranged in an array under the diffuser plate 14. The first LEDs 41 are selected from a group consisting of a red LED, a yellow LED, and a blue LED. The light passing through the diffuser plate and emitted from the yellow, red, and blue LED has an illuminance in a range of 1,000-3,500 lux, 6,000-9,500 lux, and 3,000-7,000 lux, respectively.

The controller 30 is electrically connected with the first illuminant module 40 and a power module 20, and includes: a charge socket 33 which provides an electrical connection between the power module 20 and the controller module 30; a power switch 31 mounted on the surface of the casing 10 to control power output of the power module 20; and a mode switch 32 mounted on the surface of the casing 10 to turn on the first illuminant module 40 or the second illuminant module 50.

The power module 20 can be an external power supply or is placed in the deposition space of the casing 10. When the power module 20 is placed in the deposition space of the casing 10, the power module 20 can contain rechargeable or dry batteries or microbatteries for power supply.

Accordingly, in the photo-stimulation device, red or yellow LEDs that emit light in a specific range of illuminance are employed to stimulate fibroblasts and collagen synthesis and to promote blood circulation as well as speed up removal of dead cells. Alternatively, blue LEDs that emit light in a specific range of illuminance are employed to inhibit or kill P. acnes or reduce and suppress melanin synthesis in melanocytes.

### EXAMPLE 2

The photo-stimulation device of Example 1 was used to illuminate human fibroblasts. The influence of light illumination on the fibroblasts was studied. In the present example, LEDs used in the illuminant modules of the photo-stimulation device emitted red light at 9,250 lux.

First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added into each well. The cells were illuminated by the red-light (9,250 lux) photo-stimulation device of Example 1 for 5, 10, 15, 30, 45, 60, and 90 minutes. Then, total PBS in the well was removed and DMEM (0.5 ml) was added into each well. The cells were incubated for another 24 hours.

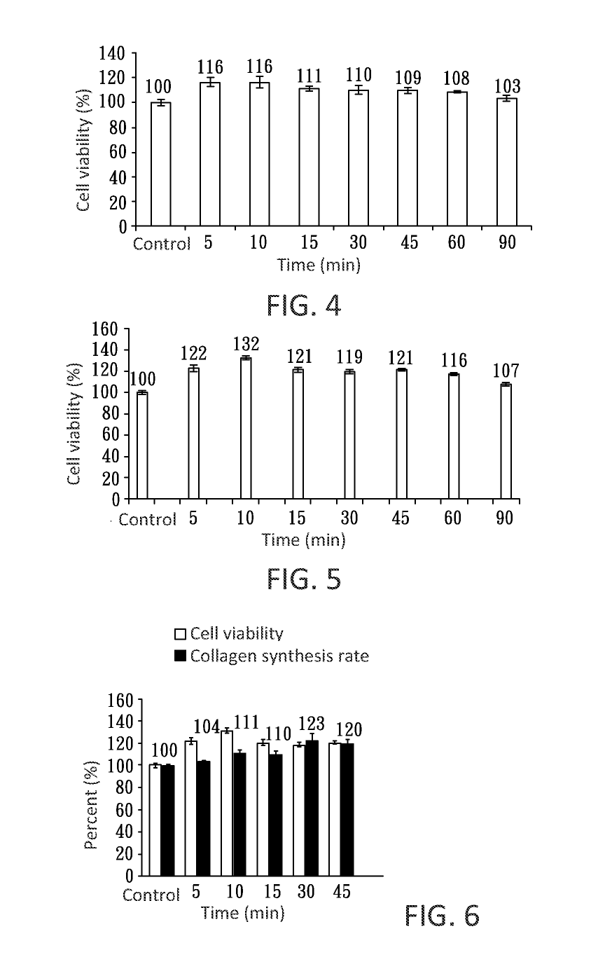

The culture medium in each well was replaced with flash DMEM (0.5 ml) and MTT reagent (0.125 ml) was added into each well. Then, the cells were incubated in an incubator (5% CO₂, 37° C.) for 4 hours. The culture media were totally collected and formazan (dissolved in DMSO, 0.5 ml) was added into the collected media. After reaction, the mixtures (0.2 ml) were analyzed in a 96-well plate by an ELISA Reader (SpectraMax M2) and absorbance thereof was measured at 570 nm. The cell viability was calculated according to the following equation where the control referred to cells that were not illuminated by the photo-stimulation device. The results are shown in FIG. 4.

Cell viability (%)=(illuminated OD₅₇₀/control OD₅₇₀)×100%

As shown in FIG. 4, the viabilities of the cells illuminated with red light at 9,250 lux for 5, 10, 15, 30, 45, 60, and 90 minutes are 116%, 116%, 111%, 110%, 109%, 108%, and 103%, respectively. All the results are shown with the average data of independently triplicate experiments. If human error is at ±10%, the increase of the cell viabilities (compared with the control) after illumination for 5-30 minutes is higher than 10%. These results indicate that the viability of the human fibroblasts is increased by illumination of red light LEDs at 9,250 lux for 5-30 minutes. In addition, illumination of red light at 9,250 does not reduce the cell viability. Therefore, the use of red light LEDs at 9,250 lux is able to meet the requirement of safety in the treatment.

### EXAMPLE 3

Based on the results of Example 2, it is understood that the use of red light illumination at 9,250 lux for 5-30 minutes can promote the viability of human fibroblasts and all of the time courses of light illumination also meet the requirement of safety in the treatment. In the present example, light illumination was performed twice and the illuminance of red light LEDs was reduced to 7,800 lux for the test of cell viability.

First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added into each well. The cells were illuminated by the red-light (7,800 lux) photo-stimulation device of Example 1 for 5, 10, 15, 30, 45, 60, and 90 minutes. Then, total PBS in the well was removed and DMEM (0.5 ml) was added into each well. The cells were incubated for another 24 hours and then the light illumination was carried out on the cells again.

The culture medium in each well was replaced with flash DMEM (0.5 ml) and MTT reagent (0.125 ml) was added in each well. Then, the cells were incubated in an incubator (5% CO₂, 37° C.) for 4 hours. The culture media were totally collected and formazan (dissolved in DMSO, 0.5 ml) was added into the collected media. After reaction, the mixtures (0.2 ml) were analyzed in a 96-well plate by an ELISA Reader (SpectraMax M2) and absorbance thereof at 570 nm was measured. The cell viability was calculated according to the following equation where the control referred to cells that were not illuminated by the photo-stimulation device. The results are shown in FIG. 5.

Cell viability (%)=(illuminated OD₅₇₀/control OD₅₇₀)×100%

As shown in FIG. 5, the viabilities of the cells illuminated with red light at 7,800 lux for 5, 10, 15, 30, 45, 60, and 90 minutes are 122%, 132%, 121%, 119%, 121%, 116%, and 107%, respectively.

All the results are shown with the average data of independently triplicate experiments. If human error is at ±10%, the increase of the cell viabilities (compared with the control) after illumination for 5-60 minutes is higher than 10%. These results indicate that the viability of the human fibroblasts is significantly increased by illumination of red light LEDs at 7,800 lux for 5-60 minutes. Among these time courses, light illumination for 5-45 minutes results in the most significant benefit.

### EXAMPLE 4

The photo-stimulation device of Example 1 was used to illuminate human fibroblasts. The influence of light illumination on the collagen synthesis of the fibroblasts was studied. In the present example, LEDs used in the illuminant modules of the photo-stimulation device emitted red light at 7,800 lux.

First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added into each well. The cells were illuminated for 5, 10, 15, 30, and 45 minutes by the red-light (7,800 lux) photo-stimulation device of Example 1. Then, total PBS in the well was removed and DMEM (0.5 ml) was added into each well. The cells were incubated for another 24 hours and then the light illumination was carried out on the cells again.

Subsequently, the culture media were totally collected in Eppendorf tubes (1.5 ml). An aqueous solution of acetic acid (0.5 M, 0.5 ml, 4° C.) was added to each well and stood for 20 minutes to dissolve collagen. The solution of each well was collected in an Eppendorf tube. Then, acid neutralizing reagent (50 μl, Biocolor) and isolation & concentration reagent (4° C., 100 μl, Biocolor) were added to the Eppendorf tubes in sequence. The mixture stood at 4° C. overnight, and was then centrifugated at 12000 rpm for 10 minutes. The supernatant was removed. Then, sircol dye reagent (1 ml, Biocolor) was added into the tubes. The tubes were sonicated for 30 minutes and centrifugated at 12,000 rpm for 10 minutes. The supernatant was removed. Subsequently, acid-salt wash reagent (4° C., 750 μl, Biocolor) was added into the tubes. The tubes were centrifugated at 12,000 rpm for 10 minutes. The supernatant was removed. Then, alkali reagent (250 μl, Biocolor) was added into the tubes. The mixture (200 μl) of each tube was taken out and added to each well of a 96-well plate. The absorbance of the mixtures at 570 nm was measured.

Collagen synthesis rate (%)=(Collagen synthesis after illumination/Collagen synthesis of control)×100%

In the equation, the control referred to cells that were not illuminated by the photo-stimulation device. The results are shown in FIG. 6 which also shows cell viability of the fibroblasts that is analyzed according to the manner of Example 2.

As shown in FIG. 6, the collagen synthesis rate of the fibroblasts illuminated with red light at 7,800 lux for 30 minutes is 123%. This result indicates red light illumination at 7,800 lux is able to promote collagen synthesis of fibroblasts. Especially, light illumination for 30 minutes results in the most significant effect.

### EXAMPLE 5

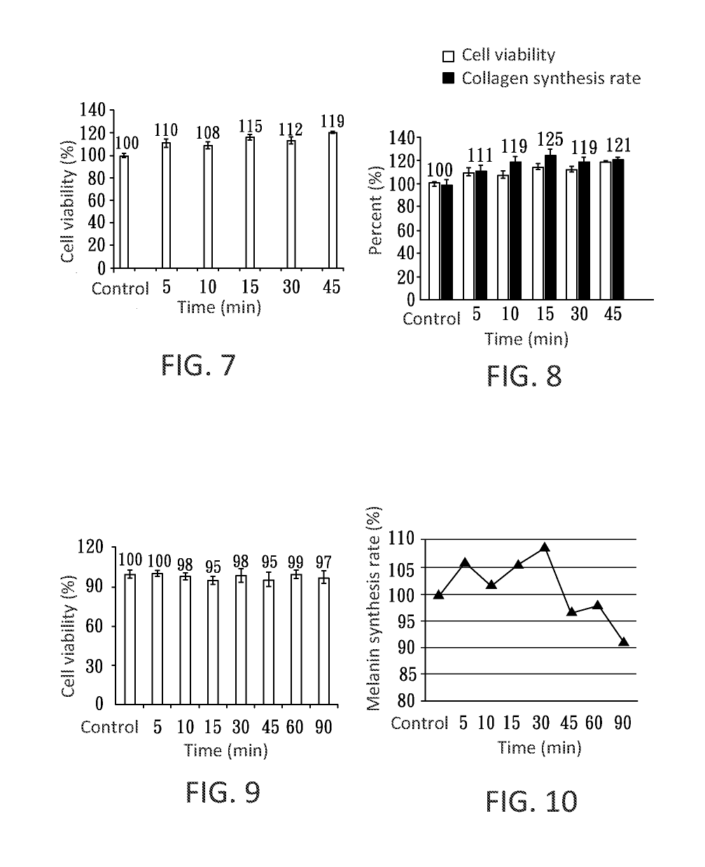

The photo-stimulation device of Example 1 was used to illuminate human fibroblasts. The influence of light illumination on the viability of the fibroblasts was studied. In the present example, LEDs used in the illuminant modules of the photo-stimulation device emitted yellow light at 2,290 lux. In addition, the viability of the fibroblasts was analyzed in the manner described in Example 3 and the fibroblasts were illuminated for 5, 10, 15, 30, and 45 minutes. The results are shown in FIG. 7.

As shown in FIG. 7, the viability of the fibroblasts illuminated with yellow light at 2,290 lux for 15 minutes is 115%. This result indicates yellow light illumination at 2,290 lux is able to promote viability of fibroblasts. Especially, light illumination for 10-45 minutes results in more significant effects.

### EXAMPLE 6

The photo-stimulation device of Example 1 was used to illuminate human fibroblasts. The influence of light illumination on the collagen synthesis of the fibroblasts was studied. In the present example, LEDs used in the illuminant modules of the photo-stimulation device emitted yellow light at 2,290 lux. In addition, the collagen synthesis of the fibroblasts was analyzed in the manner described in Example 4. The results are shown in FIG. 8 which also shows cell viability of the fibroblasts that is analyzed according to the manner of Example 5.

As shown in FIG. 8, the viability of the fibroblasts illuminated with yellow light at 2,290 lux for 15 minutes is 125% and cytotoxic effect does not occur. This result indicates yellow light illumination at 2,290 lux is able to promote collagen synthesis of fibroblasts. Especially, light illumination for 10-45 minutes results in more significant effects.

### EXAMPLE 7

The photo-stimulation device of Example 1 was used to illuminate human melanocytes. The influence of light illumination on the viability of the melanocytes was studied. In the present example, LEDs used in the illuminant modules of the photo-stimulation device emitted blue light at 5,330 lux.

First, human melanocytes (7×10⁴ cells/well) were seeded with α-MSH in a 24-well plate. To each well was added culture medium containing 10% FBS (Hyclone). Each well of the 24-well plate contained the cells and the culture medium in a total volume of 0.5 ml and the cells therein were cultured for 24 hours in a CO₂ incubator. Subsequently, all the culture media were removed and then PBS (0.5 ml) was added into each well. The cells were illuminated for 5, 10, 15, 30, 45, 60, and 90 minutes by the blue-light (5,330 lux) photo-stimulation device (additionally equipped with electrical fans to maintain the temperature) of Example 1. Then, total PBS in the well was removed and DMEM (0.5 ml) was added into each well. The cells were incubated for another 24 hours.

Then, the culture medium in each well was replaced with flash culture medium (0.5 ml) and MTT reagent (0.125 ml) was added into each well. Then, the cells were incubated in an incubator (5% CO₂, 37° C.) for 4 hours. The culture media were totally collected and formazan (dissolved in DMSO, 0.5 ml) was added into the collected media. After reaction, the mixtures (0.2 ml) were analyzed in a 96-well plate by an ELISA Reader (SpectraMax M2) and absorbance thereof at 570 nm was measured. The cell viability was calculated according to the following equation where the control referred to cells that were not illuminated by the photo-stimulation device. The results are shown in FIG. 9.

Cell viability (%)=(illuminated OD₅₇₀/control OD₅₇₀)×100%

As shown in FIG. 9, cytotoxic effect does not occur. Compared with the control, the results are within human errors of ±10%. This indicates blue light illumination at 5,330 lux is still safe for the treatment.

### EXAMPLE 8

The photo-stimulation device of Example 1 was used to illuminate human melanocytes. The influence of light illumination on the melanin synthesis of the melanocytes was studied. In the present example, LEDs used in the illuminant modules of the photo-stimulation device emitted blue light at 5,330 lux.

First, human melanocytes (1×10⁵ cells/well) were seeded with α-MSH in a 24-well plate. To each well was added culture medium containing 10% FBS (Hyclone). Each well of the 24-well plate contained the cells and the culture medium in a total volume of 0.5 ml and the cells therein were cultured for 24 hours in a CO₂ incubator. Subsequently, all the culture media were removed and then PBS (0.5 ml) was added into each well. The cells were illuminated for 5, 10, 15, 30, 45, 60, and 90 minutes by the blue-light (5,330 lux) photo-stimulation device (additionally equipped with electrical fans to maintain the temperature) of Example 1. Then, total PBS in the well was removed and DMEM (0.5 ml) was added into each well. The cells were incubated for another 24 hours.

Then, the culture medium of each well is totally removed. The cells were harvested with Trypsin-EDTA solution (1×) and centrifugated at 1,000 rpm for 10 minutes. The supernatant was removed. Then, a NaOH aqueous solution (200 μl, 1 M) was added into the tube to dissolve melanin in the cells. Absorbance of the mixtures at 490 nm was measured to determine the amount of melanin. The results are shown in FIG. 10.

As shown in FIG. 10, the melanin synthesis rate of the cells illuminated with blue light at 5,330 lux for 5, 10, 15, 30, 45, 60, and 90 minutes are 105%, 101%, 105%, 108%, 96%, 98%, and 91%, respectively. All the results are shown with the average data of independently triplicate experiments. These results indicate that blue light illumination for 90 minutes results in the decrease (about 10%) of melanin in melanocytes.

### EXAMPLE 9

The photo-stimulation device of Example 1 was used to illuminate P. acnes. The influence of light illumination on the viability of P. acnes was studied. In the present example, LEDs used in the illuminant modules of the photo-stimulation device emitted blue light at 5,710 lux.

First, a lyophilized stock culture was taken out from a refrigerator. Streaking was performed on an agar plate. Then, a single colony was picked by a sterilized loop and then spread uniformly on another agar plate. After incubation for 48 hours, the bacterium was scraped from the agar plate and suspended in sterilized water. The suspension was adjusted to OD₆₀₀=0.1 and then diluted with the same volume of sterilized water so as to obtain a bacterium broth containing 10⁶ bacteria.

Subsequently, the broth (5 ml) was spread in each of nine Petri dishes (6 cm) and illuminated by the blue-light (5,710 lux) photo-stimulation device of Example 1 for 5, 10, 15, 30, 45, 60, and 90 minutes. Then, the illuminated broths were ten-fold serial diluted into 10⁻³, 10⁻⁴, and 10⁻⁵. The diluted broths (0.1 ml) with respective concentrations were spread on triplicate RCM agar plates (BD biosciences) and cultured under an anaerobic condition at 37° C. for 48 hours. Then, the colony number on the plate was calculated and 30-300 colony-forming units (CFUs) found on one plate were considered as an effective number of colonies.

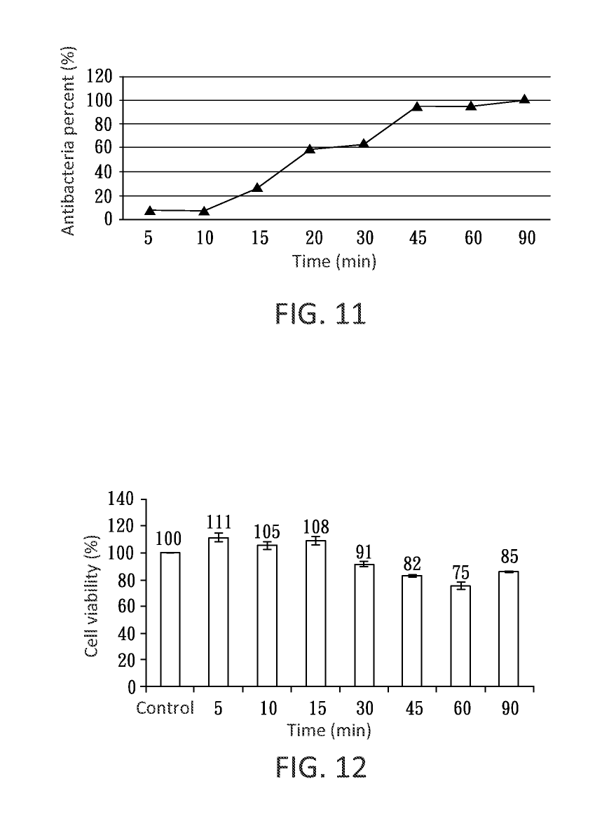

In addition, the residual broth (0.1 m) was cultured in RCM broth under an anaerobic condition at 37° C. for 48 hours. Absorbance of the broth at 600 nm was measured so that growth changes of P. acnes were observed. The results are shown in FIG. 11. As shown in FIG. 11, after illumination for 45 minutes, the inhibition of P. acnes reaches 95%. This demonstrates extremely significant inhibition.

### COMPARATIVE EXAMPLE

The photo-stimulation device was used to illuminate human fibroblasts. The influence of light illumination on the viability of the fibroblasts was studied. In the present example, LEDs used in the illuminant modules of the photo-stimulation device emitted red light at 9,890 lux. In addition, the viability of the fibroblasts was analyzed in the manner described in Example 2. The results are shown in FIG. 12.

As shown in FIG. 12, the viabilities of the cells illuminated with red light at 9,890 lux for 5, 10, 15, 30, 45, 60, and 90 minutes are 111%, 105%, 108%, 91%, 82%, 75%, and 85%, respectively. All the results are shown with the average data of independently triplicate experiments.

If human error is at ±10%, the increase of the cell viabilities (compared with the control) after illumination for 5 minutes is higher than 10%. However, the decrease of the cell viabilities (compared with the control) after illumination for 45-90 minutes is also higher than 10%. Accordingly, although red light illumination at 9,890 lux for 5 minutes is able to slightly increase cell viability of human fibroblasts, the cell viability decreases as the time course of illumination increase. When the time course reaches 30 minutes, the cell viability is lower than that of the control. When the time course exceeds 45 minutes, the cell viability is decreased more and more. Hence, illumination of red light from LEDs at 9,890 lux will incur qualms about safety.

Although the present invention has been explained in relation to its preferred embodiment, it is to be understood that many other possible modifications and variations can be made without departing from the spirit and scope of the invention as hereinafter claimed.

1-9. (canceled)

10. A photo-stimulation device, comprising:
a casing forming a deposition space and having a top surface and a lateral surface, wherein the top surface is provided with a light-output window;
a diffuser plate covering the light-output window of the casing;
a first illuminant module located in the deposition space of the casing and having a first light-emitting diode (LED) located under the diffuser plate, and the first LED selected from one of a group consisting of a red LED, a yellow LED, and a blue LED, wherein the light emitted from the yellow LED and passing through the diffuser plate has an illuminance in a range of 1,000-3,500 lux, the light emitted from the red LED and passing through the diffuser plate has an illuminance in a range of 6,000-9,500 lux, and the light emitted from the blue LED and passing through the diffuser plate has an illuminance in a range of 3,000-7,000 lux; and
a light-transmission plate covering a light-output hole provided in the lateral surface of the casing;
a second illuminant module deposed corresponding to the light-transmission plate and emitting light which passes through the light-transmission plate; and
a controller module electrically connected with the first illuminant module and a power module.

11. The photo-stimulation device of claim 10, wherein the power module is an external power supply or is placed in the deposition space of the casing.

12. The photo-stimulation device of claim 11, wherein the controller module comprises a charge socket which provides an electrical connection between the power module and the controller module.

13. The photo-stimulation device of claim 10, wherein the controller module comprises a power switch mounted on the surface of the casing to control power output of the power module.

14-15. (canceled)

16. The photo-stimulation device of claim 10, wherein the controller module comprises a mode switch mounted on the surface of the casing to turn on the first illuminant module or the second illuminant module.
